(19) United States
(12) Patent Application Publication
(10) Pub. No.: US 2024/0384206 A1
(43) Pub. Date: Nov. 21, 2024

(54) BIOACTIVE-CONTAINING GRANULES

(71) Applicant: Danisco US Inc., Palo Alto, CA (US)

(72) Inventors: Samuel A Maurer, San Francisco, CA (US); Joseph C McAuliffe, Sunnyvale, CA (US); Kurinji Krishnamoorthy, Leiden (NL); Dana Wong, San Franciso, CA (US)

(21) Appl. No.: 18/688,856

(22) PCT Filed: Sep. 13, 2022
(86) PCT No.: PCT/US2022/043281

§ 371 Date: Mar. 4, 2024

Related U.S. Application Data
(60) Provisional application No. 63/243,243, filed on Sep. 13, 2021.

(51) Int. Cl. C11D 3/386; C11D 1/72; C11D 3/00; C11D 3/12; C11D 3/22; C11D 3/37; C11D 17/00; C12N 9/28; C12N 9/54

(52) U.S. Cl. CPC C11D 3/38672; C11D 1/72; C11D 3/0068; C11D 3/1213; C11D 3/1233; C11D 3/128; C11D 3/222; C11D 3/3753; C11D 3/38609; C11D 17/0039; C12N 9/2417; C12N 9/54; C12Y 302/01001; C12Y 304/21062

(57) ABSTRACT

Disclosed herein are enzyme-containing granules, and compositions and methods related to the production and use thereof, including bioactive-containing granules that have improved features compared to one or more reference granules.

This application is a 371 of International Patent Application No. PCT/US2022/043281, filed Sep. 13, 2022, which claims the benefit of U.S. Provisional Application No. 63/243,243, filed Sep. 13, 2021, each of which is hereby incorporated by reference in its entirety.

The present compositions and methods relate to bioactive-containing granules. The granules are particularly useful in consumer and industrial products, such as detergent, animal feed, food, personal care and agricultural compositions.

### BACKGROUND

The use of proteins such as pharmaceutically important proteins, e.g., hormones, and industrially important proteins, e.g., enzymes, has continued to grow over the past decade. Today, for example, enzymes find frequent use in the animal nutrition, food, grain processing, and detergent industries, among others.

In the detergent industry, in particular, enzymes are often configured in a granular form, with an eye toward achieving one or more desirable storage and/or performance characteristics, depending upon the particular application at hand. In these regards, the industry has offered numerous developments in the granulation and coating of enzymes.

Similarly, the use of products containing live microbial cultures has also grown over the past decade. Products containing live microbial cultures find frequent use in the biopharmaceutical, probiotic, food, and agricultural industries, among others. Within these industries, delivery of the products in a granular form is often key to extending the shelf life of the microbes to acceptable levels. Similarly, the industry has offered numerous developments in the granulation and coating of live microbial products in granular form.

Notwithstanding such developments, there is a continuing need for enzyme and live microbial granules which have additional beneficial or improved characteristics.

Typically, both products containing proteins and products containing live microbes are produced via fermentation: either a fermentation of a microbe expressing a protein of interest or a fermentation of the microbial cultures(s) to be delivered in the finished product. Often, malodorous compounds such as organic acids, sulfides, and amines can be produced during these fermentation processes. In industries such as detergents, food, pharmaceuticals, and agriculture, the presence of malodorous compounds in the finished granular product is undesirable for the consumer. Thus, methods of controlling the malodor that is perceived by smelling an enzyme granule are beneficial to the manufacture of bioactive granules.

### SUMMARY

One embodiment is directed to granules comprising a core and at least one layer, wherein the granule comprises at least one bioactive and at least one odor-controlling layer. In one embodiment, the disclosure provides a granule comprising a core and at least one layer, where the granule comprises: a) a first layer comprising about 0.1% to about 40% wt/wt of bioactive enzyme or microorganism; and b) an odor-controlling coating layer surrounding the first layer, where the odor-controlling coating layer comprises between about 3-10% wt/wt of a water-soluble polymer, between about 0.1%-5% wt/wt of a nonionic surfactant, and between about 0.1-10% wt/wt of an odor-controlling compound. In some embodiments, the enzyme is selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, DNase or nuclease, endo-beta-1, 4-glucanases, endo-beta-mannanases, esterases, exo-mannanases, galactanases, glucoamylases, hemicellulases, hyaluronidases, keratinases, laccases, lactases, ligninases, lipases, lipoxygenases, lysozymes, mannanases, oxidases, pectate lyases, pectin acetyl esterases, pectinases, pentosanases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, xylosidases, metalloproteases, nucleases, serine proteases, and combinations thereof.

Also provided are granules comprising a core and at least odor-controlling layer substantially surrounding the core, wherein the granule comprises: a) a bioactive matrix core comprising about 0.1% to about 40% wt/wt of bioactive enzyme or microorganism; and b) an odor-controlling coating layer substantially surrounding the bioactive matrix core, where the odor-controlling coating layer comprises between about 3-10% wt/wt of a water soluble polymer, between about 0.1%-5% wt/wt of a nonionic surfactant, and between about 0.1-10% wt/wt of an odor-controlling compound.

In additional embodiments, the disclosure provides methods of cleaning a surface comprising: a) contacting a surface with a composition comprising a granule comprising i) a first layer comprising about 0.1% to about 40% wt/wt of bioactive enzyme or microorganism; and an odor-controlling coating layer surrounding the first layer, where the odor-controlling coating layer comprises between about 3-10% wt/wt of a water-soluble polymer, between about 0.1%-5% wt/wt of a nonionic surfactant, and between about 0.1-10% wt/wt of an odor-controlling compound, or ii) a bioactive matrix core comprising about 0.1% to about 40% wt/wt of bioactive enzyme or microorganism; and an odor-controlling coating layer substantially surrounding the bioactive matrix core, where the odor-controlling coating layer comprises between about 3-10% wt/wt of a water soluble polymer, between about 0.1%-5% wt/wt of a nonionic surfactant, and between about 0.1-10% wt/wt of an odor-controlling compound; and b) rinsing the surface.

In some embodiments, the surface contacted in the methods provided herein is selected from a dish or hard surface.

### DESCRIPTION OF THE FIGURES

FIG. 1 provides the results of one embodiment of the disclosure for odor reduction for protease-containing granules, relative to Granule A presented as a control granule.

FIG. 2 provides the results of one embodiment of the disclosure for odor reduction for amylase-containing granules, relative to Granule D presented as a control granule.

### DESCRIPTION

The present disclosure provides bioactive granules comprising an odor-controlling layer containing an odor-controlling compound and compositions containing such granules. The present disclosure also provides methods using such granules and compositions for cleaning surfaces (e.g. fabric surfaces or hard surfaces) and methods for reducing malodors associated with granules comprising enzymes or other fermentation products.

Prior to describing embodiments of present compositions and methods, the following terms are defined.

Unless defined otherwise herein, all technical and scientific terms used herein have the same meaning as commonly understood by one of ordinary skill in the art to which this invention pertains. Although any methods and materials similar or equivalent to those described herein find use in the practice of the present invention, the preferred methods and materials are described herein. Accordingly, the terms defined immediately below are more fully described by reference to the specification as a whole. Also, as used herein, the singular terms “a,” “an,” and “the” include the plural reference unless the context clearly indicates otherwise. It is to be understood that this invention is not limited to the particular methodology, protocols, and reagents described, as these may vary, depending upon the context they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the term “granule” refers to a small compact particle of a substance. The particle comprises a core with one or more optional coating layers.

As used herein, the term “core” is interchangeable with the term “seed” and comprises the unitary inner part of a granule upon which additional coatings or layers can be applied. A core may comprise a single material such as a salt or sugar crystal or may be composed of a mixture of materials. A core may be inert or may comprise on or more bioactives, either as pure bioactive or bioactive mixed or embedded within a matrix of inert materials.

As used herein, the term “multi-layered granule” refers to a composition comprising a core and at least one coating layer. The core may be an inert core or a matrix core.

As used herein, the term “coating layer” and “layer” are interchangeable. The coating layer(s) generally encapsulates the core in order to form a substantially continuous layer so that the core surface has few or no uncoated areas. The materials (e.g., the agents, components and enzyme detailed herein) used in the granule and/or multi-layered granule are suitable for the use in foods and/or animal feeds. The materials can be food grade or feed grade.

As used herein, the term “bioactive coating layer” or “bioactive layer” refers to a granule layer that comprises at least one enzyme or microorganism, or combinations of at least one enzyme and one microorganism.

As used herein, the term “enzyme coating layer” or “enzyme layer” refers to an enzyme layer that comprises at least one enzyme.

As used herein, the term “microbial coating layer” or “microbial layer” refers to a layer that comprises at least one live microorganism such as, but not limited to, a bacterial and/or fungal microorganism at any developmental stage (e.g. a vegetative cell, spore, and/or any mix thereof).

As used herein, the term “bioactive matrix core” refers to a granule core comprising at least one enzyme or microorganism, or combinations of at least one enzyme and one microorganism. A bioactive matrix core may further comprise fermentation solids and excipients, such as binders and fillers. The bioactive is dispersed or dissolved throughout the matrix core and is not layered upon a unitary inert core devoid of a bioactive.

As used herein, the term “enzyme matrix core” refers to a granule core comprising enzyme. An enzyme matrix core may further comprise fermentation solids and excipients, such as binders and fillers. The active enzyme is dispersed or dissolved throughout the enzyme matrix core and is not layered upon a unitary inert core devoid of enzyme.

As used herein, the term “microbial matrix core” refers to a granule core comprising that comprises at least one live microorganism such as, but not limited to, a bacterial and/or fungal microorganism at any developmental stage (e.g. a vegetative cell, spore, and/or any mix thereof). A microbial matrix core may further comprise fermentation solids and excipients, such as binders and fillers. The microbial cultures are dispersed or dissolved throughout the microbial matrix core and are not layered upon a unitary inert core devoid of microbial cultures.

As used here, “continuous,” with respect to a granule layer, means uninterrupted by breaks, cracks, or holes, such that that the properties of the contiguous portions of the coating control the properties of a coating, as opposed to breaks, cracks, or holes in the coating. The continuity of a coating can be assessed by observing a representative sample of granules under scanning electron microscope (SEM).

As used herein, “weight percent,” “weight fraction,” “mass fraction” or simply “fraction” refers to the relative amount of mass on a % wt/wt or fractional wt/wt basis, for example, the relative amount of mass of a coating compared to the mass of an entire granule. As used herein, the term “fermentation solids” refers to dried or partially dried solids derived from a microbial fermentation broth that is processed so as to recover one or more useful bioactives of interest, such as an enzyme or live microbe. Fermentation solids can be derived from whole cell broth obtained directly from a fermenter, from clarified broth with cells removed by filtration or centrifugation, concentrated, e.g., by ultrafiltration or evaporation, or purified to varying degrees, e.g., by chromatography, precipitation or crystallization. Fermentation solids can thereby include impurities other than the enzyme actives, such as inactive protein, peptides. amino acids, polysaccharides, sugars, salts and other residual compounds formed during fermentation and downstream processing. Fermentation solids may also consist of solid material containing live microbes from a fermentation that are dried to produce a bioactive product delivering a live microbe, for example in a probiotic or agricultural application. Fermentation solids may also comprise some residual free or bound water remaining after a drying or granulation process. Fermentation solids do not include excipients, which are defined separately (infra).

As used herein, “payload” refers to the mass fraction of a material of interest within a granule. The material of interest, within the scope of this invention, is either fermentation solids in aggregate, or only enzyme, depending on the context specified. Payload is expressed as % wt/wt solids of either fermentation solids or enzyme relative to the total mass of the granule. In this manner, one can also refer to “enzyme payload”. “bioactive payload”, or “fermentation solids payload.”

As used herein, the term “excipients” refers to solids added to fermentation solids during or after processing but prior to drying or granulation, in order to improve the stability, handling, or physical properties of the resulting dry granules. In this use, excipients are not counted within the enzyme payload or fermentation solids payload of a granule. Excipients include, but are not limited to: stabilizers, binders, viscosity modifiers, surfactants, fillers, lubricants, desiccants, humectants, pigments, odor-controlling compounds, and the like.

As used herein, the term “about” refers to ±15% to the referenced value.

As used herein, “cleaning compositions” and “cleaning formulations” refer to compositions that may be used for the removal of undesired compounds from items to be cleaned, such as fabric, dishes, contact lenses, other solid substrates, hair (shampoos), skin (soaps and creams), teeth (mouthwashes, toothpastes), etc. The term encompasses any materials/compounds selected for the particular type of cleaning composition desired. The specific selection of cleaning composition materials are readily made by considering the surface, item or fabric to be cleaned, and the desired form of the composition for the cleaning conditions during use.

As used herein, the terms “detergent composition” and “detergent formulation” are used in reference to mixtures which are intended for use in a wash medium for the cleaning of soiled objects. In some preferred embodiments, the term is used in reference to laundering fabrics and/or garments (e.g., “laundry detergents”). In alternative embodiments, the term refers to other detergents, such as those used to clean hard surfaces such as dishes, cutlery, etc. (e.g., “dishwashing detergents”).

As used herein, the term “hard surface” refers to any article having a hard surface including floors, tables, walls, roofs etc. as well as surfaces of hard objects such as cars (car wash), ship hulls, dishes (dishware), medical instruments, pipes, reservoirs, or holding tanks. The term “hard surface” includes also the surfaces of flexible yet firm objects such as the insides of bendable tubing and supply lines or the surfaces of deformable holding tanks or vessels. The term “hard surface” includes also the surfaces in the interior of washing machines, such as the interior of laundry washing machines or dishwashing machines, this includes soap intake box, walls, windows, baskets, racks, nozzles, pumps, sump, filters, pipelines, tubes, joints, seals, gaskets, fittings, impellers, drums, drains, traps, coin traps inlet and outlets. The term hard surface does not encompass textile or fabric.

As used herein, “personal care products” means products used in the cleaning, bleaching and/or disinfecting of hair, skin, scalp, and teeth, including, but not limited to shampoos, body lotion, shower gels, topical moisturizers, toothpaste, and/or other topical cleansers. In some particularly preferred embodiments, these products are utilized on humans, while in other embodiments, these products find use with non-human animals (e.g., in veterinary applications).

The following abbreviations may be used in the description. Definitions are also provided as needed throughout the description.
° C. degree Centigrade
Tₘ melting temperature
H₂O Water
a_{w} water activity
Min Minute
Hr Hour
w/w weight/weight
wt % weight percent
g or gm Grams
mM Millimolar
mg Milligrams
Mg Micrograms
mL and ml Milliliters
μL and μl Microliters

### Enzyme Granules

The granules provided herein are generally made up of a core, a bioactive layer containing one or more bioactives, and an odor-controlling coating layer. In some embodiments, the granule comprises between about 0.1-40% by total weight of the one or more enzymes, and an odor-controlling coating layer comprising between about 0.1-5% by total weight of an odor-controlling compound. In some embodiments, the odor-controlling layer comprises between about 2.0-4.0% wt/wt of an odor-controlling compound.

In some embodiments, the disclosure provides a population of bioactive-containing granules, where at least about 50%, 60%, 70%, 80%, 85%, 90%, or 95% of the granules have a diameter of about 150 μm to about 300 μm, about 150 μm to about 350 μm, about 150 μm to about 355 μm, about 180 μm to about 300 μm, about 180 μm to about 350 μm, about 210 μm to about 350 μm, about 212 μm to about 355 μm, about 180 μm to about 355 μm, about 210 μm to about 400 μm, about 210 μm to about 500 μm, or about 210 μm to about 595 μm. In some embodiments, at least about 50%, 60%, 70%, 80%, 85%, 90%, or 95% of the granules have a diameter of any of about 150, 160, 170, 180, 190, 200, or 210 μm to any of about 250, 260, 270, 280, 290, 300, 310, 320, 330, 340, 350, 355, 375, 400, 420, 440, 470 or 500 μm.

### Odor-Controlling Coating Layer

In one embodiment, the granules provided herein comprise at least one odor-controlling coating layer that comprises at least one odor-controlling compound. A variety of compounds can be used for odor absorption, including aluminosilicates (e.g. zeolites), clays, saccharides, functionalized saccharides, diatomaceous earth, metal oxides, and metal carbonates. Odor-controlling compounds typically reduce malodor perceived in a product by interacting either physically or chemically with malodorous compounds that are present in the gas phase. By sequestering these compounds and preventing their release into the gas phase, the level of malodor perceived by a customer smelling the product is reduced. Odor-controlling compounds typically have a high specific surface area, either due to a fine particle size or a channelled microstructure, thereby allowing numerous surface sites for molecular interaction with volatile malodorous compounds in the gas phase.

In some embodiments, the granule provided herein comprises an odor-controlling compound in an amount of between about 0.1-10% wt/wt, between about 0.5-7% wt/wt, or between about 1.0-5.0% wt/wt of an odor-controlling compound.

In some embodiments, the odor-controlling compound is an aluminosilicate, such as a zeolite. In some embodiments, the odor-controlling layer also comprises additional components, such as a water-soluble polymer and a surfactant. Surfactants for use in the odor-controlling layer can be selected from the group of cationic surfactants, nonionic surfactants, anionic, and amphoteric surfactants, and mixtures thereof.

In some embodiments, the odor-controlling layer of the granule is the outer layer of the granule. In some embodiments, the outer layer comprises a zeolite, a water-soluble polymer, and a nonionic surfactant.

Zeolites are generally described as crystalline, hydrated aluminosilicates with a three-dimensional framework structure constructed of SiO₄ and AlO₄ tetrahedra linked through oxygen bridges. The tetrahedra of SiO₄ and AlO₄ are the primary building blocks; the combination of which leads to the so-called secondary building units such as 4-, 5-, and 6-rings, double 4-, 5-, and 6-rings, and so on. Depending on the structure type, zeolites contain regular channels or interlinked voids whose aperture diameters are in the microporous range, i.e. below 2 nm. These pores contain water molecules and the cations necessary to balance the negative charge of the framework. The cations, which are mobile and can be exchanged, are mainly alkali metal or alkaline-earth metal ions.

The International Union of Pure and Applied Chemistry (IUPAC) provided guidelines for specifying the chemical formula for zeolites (sec, e.g., McCusker, L.B. et al. (2003) “Nomenclature of structural and compositional characteristics of ordered microporous and mesoporous materials with inorganic hosts (IUPAC recommendations 2001)” MicroporousMesoporousMater. 58:3.). In the simplest form, a general formula can be given as:
   |M_{x/n}(H₂O)_{y}|[AlₓSi₍ₜ₋ₓ₎O₂ₜ]−LTA, wherein
the guest species are listed between the braces, i.e., “||,” and
the host framework is listed between the brackets, i.e., “[],”
M represents a charge-balancing cation such as Na, K, Ca or Mg,
x is the number of framework Al atoms in the unit cell,
n is the cation charge,
y is the number of adsorbed water molecules,
t is the total number of framework tetrahedral atoms in the unit cell (Al+Si), and
LTA, herein entered as an example, is the code or the framework type.

In general, any zeolite may be used in the granules described herein. The preferred zeolites of the present disclosure include Zeolite-Beta Hydrogen, Zeolite-Beta Ammonium, Erionite, Zeolite A, Zeolite P, Zeolite MAP, Zeolite X, Zeolite Y, Mordenite, Zeocros E110, and Zeocros CG180.

In some embodiments, the odor-controlling coating layer further comprises a polymer, a surfactant, and optionally additional components.

In some embodiments, the odor-controlling coating layer does not comprise titanium dioxide.

In some embodiments, the odor-controlling coating layer is coated over the bioactive layer. In another embodiment, the odor-controlling layer is coated over a barrier layer, which is coated over the bioactive layer.

In some embodiments, the odor-controlling coating layer comprises an odor-controlling saccharide or functionalized saccharide, such as alpha-cyclodextrin, beta-cyclodextrin, gamma-cyclodextrin, starch, cellulose, methylcellulose, or hydroxypropylmethylcellulose.

In some embodiments, the odor-controlling coating layer comprises an odor-controlling clay, such as kaolinite, bentonite, montmorillonite, atapulgite, illite, bentonite, halloysite or diatomite (diatomaceous earth).

In some embodiments, the odor-controlling coating layer comprises an odor-controlling metal oxide, such as titanium dioxide, magnesium oxide, iron (III) oxide, calcium oxide, or silicon dioxide.

In some embodiments, the odor-controlling coating layer comprises an odor-controlling metal carbonate, such as calcium carbonate, magnesium carbonate, or sodium hydrogen carbonate.

In some embodiments, the odor-controlling coating layer comprises a metal-organic framework, such as MOF-5, MOF-177, or MOF-199.

### Enzyme Layer

In some embodiments, the granule of the present disclosure contains an enzyme layer containing one or more enzymes. The enzyme layer may also contain one or more of a polymer, a sugar, a starch, a salt, or a surfactant.

The present granules, compositions and methods are applicable to many different enzymes. Exemplary enzymes include acyl transferases, α-amylases, β-amylases, arabinosidases, aryl esterases, carrageenases, catalases, cellobiohydrolases, cellulases, chondroitinases, cutinases, nucleases (such as DNases and RNases), endo-β-1,4-glucanases, endo-beta-mannanases, esterases, exo-mannanases, galactanases, α-galactosidases, β-galactosidases, β-glucanases, glucoamylases, hemicellulases, hyaluronidases, keratinases, laccases, lactases, ligninases, lipases, lipoxygenases, mannanases, oxidases, oxidoreductases, pectate lyases, pectin acetyl esterases, pectinases, pentosanases, perhydrolases, peroxidases, peroxygenases, phenoloxidases, phosphatases, phospholipases, phytases, polygalacturonases, proteases, pullulanases, reductases, rhamnogalacturonases, tannases, transglutaminases, xylan acetyl-esterases, xylanases, xyloglucanases, xylosidases, metalloproteases, proteases, and combinations, thereof.

Examples of proteases include but are not limited to subtilisins, such as those derived from Bacillus (e.g., subtilisin, lentus, amyloliquefaciens, subtilisin Carlsberg, subtilisin 309, subtilisin 147 and subtilisin 168), including variants as described in, e.g., U.S. Pat. Nos. RE 34,606, U.S. Pat. Nos. 5,955,340, 5,700,676, 6,312,936, and 6,482,628, all of which are incorporated herein by reference. Additional proteases include trypsin (e.g., of porcine or bovine origin) and the Fusarium protease described in WO 89/06270. In some embodiments the protease is one or more of MAXATASER, MAXACAL™, MAXAPEM™, OPTICLEAN®, OPTIMASER, PROPERASE®, PURAFECT®, PURAFECT® OXP, PURAMAX™, EXCELLASE™ PURAFAST™, EXCELLENZ® P, and EFFECTENZ® P (DuPont Industrial Biosciences); ALCALASER, SAVINASER, PRIMASER, DURAZYM™, POLARZYMER, OVOZYMER, KANNASER, LIQUANASER, NEUTRASER, RELASER, ESPERASER, BLAZER (Novozymes); BLAP™ and BLAP™ variants (Henkel Kommanditgesellschaft auf Aktien, Duesseldorf, Germany), and KAP (B. alkalophilus subtilisin; Kao Corp., Tokyo, Japan). Additional proteases are described in WO95/23221, WO 92/21760, WO 09/149200, WO 09/149144, WO 09/149145, WO 11/072099, WO 10/056640, WO 10/056653, WO 11/140364, WO 12/151534, WO2016/205755 U.S. Pat. Publ. No. 2008/0090747, and U.S. Pat. Nos. 5,801,039, 5,340,735, 5,500,364, 5,855,625, US RE 34,606, U.S. Pat. Nos. 5,955,340, 5,700,676, 6,312,936, and 6,482,628.

Proteases include neutral metalloproteases including those described in WO 07/044993 and WO 09/058661. Other exemplary metalloproteases include nprE, the recombinant form of neutral metalloprotease expressed in Bacillus subtilis (sec e.g., WO 07/044993), and PMN, the purified neutral metalloprotease from Bacillus amyloliquefacients.

Lipases include, but are not limited to Humicola lanuginosa lipase (see e.g., EP 258 068, and EP 305 216), Rhizomucor miehei lipase (See e.g., EP 238 023), Candida lipase, such as C. antarctica lipase (e.g., the C. antarctica lipase A or B; See e.g., EP 214 761), Pseudomonaslipases such as P. alcaligenes lipase and P. pseudoalcaligenes lipase (sec e.g., EP 218 272), P. cepacia lipase (Sec e.g., EP 331 376), P. stutzeri lipase (Sec e.g., GB 1,372,034), P. fluorescenslipase, Bacillus lipase (e.g., B. subtilis lipase (Dartois et al. (1993) Biochem. Biophys. Acta 1131:253-260); B. stearothermophilus lipase (see e.g., JP 64/744992); and B. pumilus lipase (sec e.g., WO 91/16422)).

Additional lipases include Penicillium camembertii lipase (Yamaguchi et al. (1991) Gene 103:61-67), Geotricum candidum lipase (Sec, Schimada et al. (1989) J. Biochem. 106:383-388), and various Rhizopus lipases such as R. delemar lipase (Hass et al. (1991) Gene 109:117-113), a R. niveus lipase (Kugimiya et al. (1992) Biosci. Biotech. Biochem. 56:716-719) and R. oryzae lipase. Additional lipases are the cutinase derived from Pseudomonas mendocina (Sec, WO 88/09367), and the cutinase derived from Fusarium solani pisi (WO 90/09446). Various lipases are described in WO 11/111143, WO 10/065455, WO 11/084412, WO 10/107560, WO 11/084417, WO 11/084599, WO 11/150157, and WO 13/033318. In some embodiments the lipase is one or more of MI LIPASE™, LUMA FAST™, LIPOMAX™ and PREFERENZ® L 100 (DuPont Industrial Biosciences); LIPEX®, LIPOLASER and LIPOLASER ULTRA (Novozymes); and LIPASE P™ “Amano” (Amano Pharmaceutical Co. Ltd., Japan).

Amylases include, but are not limited to those of bacterial or fungal origin, or even mammalian origin. Numerous suitable are described in W09510603, WO9526397, WO9623874, WO9623873, WO9741213, WO9919467, WO0060060, WO0029560, WO9923211, WO9946399, WO0060058, WO0060059, WO9942567, WO0114532, WO02092797, WO0166712, WO0188107, WO0196537, WO0210355, WO9402597, WO0231124, WO9943793, WO9943794, WO2004113551, WO2005001064, WO2005003311, WO0164852, WO2006063594, WO2006066594, WO2006066596, WO2006012899, WO2008092919, WO2008000825, WO2005018336, WO2005066338, WO2009140504, WO2005019443, WO2010091221, WO2010088447, WO0134784, WO2006012902, WO2006031554, WO2006136161, WO2008101894, WO2010059413, WO2011098531, WO2011080352, WO2011080353, WO2011080354, WO2011082425, WO2011082429, WO2011076123, WO2011087836, WO2011076897, WO94183314, WO9535382, WO9909183, WO9826078, WO9902702, WO9743424, WO9929876, WO9100353, WO9605295, WO9630481, WO9710342, WO2008088493, WO2009149419, WO2009061381, WO2009100102, WO2010104675, WO2010117511, WO2010115021, WO2013184577, WO9418314, WO2008112459, WO2013063460, WO10115028, WO2009061380, WO2009100102, WO2014099523, WO2015077126A1, WO2013184577, WO2014164777, PCT/US12/70334, PCT/US13/74282, PCT/CN2013/077294, PCT/CN2013/077134, PCT/CN2013/077137, PCT/CN2013/077142, PCT/CN2012/087135, PCT/US12/62209, PCT/CN2013/084808, PCT/CN2013/084809, and PCT/US14/23458. Commercially available amylases include, but are not limited to one or more of DURAMYL®, TERMAMYL®, FUNGAMYL®, STAINZYMER STAINZYME PLUS®, STAINZYME ULTRA®, AMPLIFY®, ACHIEVE ALPHA® and BAN™ (Novozymes), as well as POWERASE™, RAPIDASE® and MAXAMYL® P, PREFERENZ® S100, PREFERENZ® S110, and PREFERENZ® S1000 (DuPont Industrial Biosciences).

Cellulases include but are not limited to those having color care benefits (see e.g., EP 0 495 257). Examples include Humicola insolens cellulases (See e.g., U.S. Pat. No. 4,435,307) and commercially available cellulases such as CELLUZYME®, CAREZYME® (Novozymes), and KAC-500(B)™ (Kao Corporation), and PRIMAFAST® GOLD, REVITALENZ® (DuPont). In some embodiments, cellulases are incorporated as portions or fragments of mature wild-type or variant cellulases, wherein a portion of the N-terminus is deleted (See e.g., U.S. Pat. No. 5,874,276). Additional suitable cellulases include those found in WO2005054475, WO2005056787, U.S. Pat. Nos. 7,449,318, and 7,833,773.

Mannanases are described in U.S. Pat. Nos. 6,566, 114, 6,602,842, 5, 476, and 775, 6,440,991, and U.S. Patent Application No. 61/739,267, all of which are incorporated herein by reference). Commercially available include, but are not limited to MANNASTAR®, PURABRITE™, PREFERENZ® M, and MANNAWAY®.

Nucleases for use in the compositions and methods provided herein include DNases and RNases. Exemplary nucleases include, but are not limited to, those described in WO2015181287, WO2015155350, WO2016162556, WO2017162836, WO2017060475 (e.g. SEQ ID NO: 21), WO2018184816, WO2018177936, WO2018177938, WO2018/185269, WO2018185285, WO2018177203, WO2018184817, WO2019084349, WO2019084350, WO2019081721, WO2018076800, WO2018185267, WO2018185280, WO2018206553, and WO2019/086530. Other nucleases which can be used in the compositions and methods provided herein include those described in Nijland R, Hall MJ, Burgess JG (2010) Dispersal of Biofilms by Secreted, Matrix Degrading, Bacterial DNase. PLOS ONE 5(12) and Whitchurch, C.B., Tolker-Nielsen, T., Ragas, P.C., Mattick, J.S. (2002) Extracellular DNA required for bacterial biofilm formation. Science 295:1487.

In some embodiments, peroxidases are used in combination with hydrogen peroxide or a source thereof (e.g., a percarbonate, perborate or persulfate) in the compositions of the present teachings, to the extent possible. In some alternative embodiments, oxidases are used in combination with oxygen. Both types of enzymes are used for “solution bleaching” (i.e., to prevent transfer of a textile dye from a dyed fabric to another fabric when the fabrics are washed together in a wash liquor), preferably together with an enhancing agent (See e.g., WO 94/12621 and WO 95/01426). Suitable peroxidases/oxidases include, but are not limited to those of plant, bacterial or fungal origin. Chemically or genetically modified mutants are included in some embodiments.

Perhydrolases include the enzyme from Mycobacterium smegmatis. This enzyme, its enzymatic properties, its structure, and numerous variants and homologs, thereof, are described in detail in International Patent Application Publications WO 05/056782A and WO 08/063400A, and U.S. Patent Publications US2008145353 and US2007167344, which are incorporated by reference. In some embodiments, the Mycobacterium smegmatis perhydrolase, or homolog, includes the S54V substitution.

Other perhydrolases include members of the carbohydrate family esterase family 7 (CE-7 family) described in, e.g., WO2007/070609 and U.S. Patent Application Publication Nos. 2008/0176299, 2008/176783, and 2009/0005590. Members of the CE-7 family include cephalosporin C deacetylases (CAHs; E.C. 3.1.1.41) and acetyl xylan esterases (AXEs; E.C. 3.1.1.72). Members of the CE-7 esterase family share a conserved signature motif (Vincent et al., J. Mol. Biol., 330:593-606 (2003)).

Other perhydrolase enzymes include those from Sinorhizobium meliloti, Mesorhizobium loti, Moraxella bovis, Agrobacterium tumefaciens, or Prosthecobacter dejongeii(WO2005056782), Pseudomonas mendocina (U.S. Pat. No. 5,389,536), or Pseudomonas putida (U.S. Pat. Nos. 5,030,240 and 5,108,457).

The enzyme layer may also optionally include one or more other components in addition to the one or more enzyme(s). Such non-enzyme components include, but are not limited to, polymers (e.g., polyvinyl alcohol, polyethylene glycol), sugars (e.g., sucrose, saccharose, glucose, fructose, galactose, maltodextrin), starches (e.g., corn starch, wheat starch, tapioca starch, potato starch, chemically or physically modified starch), dextrins, antifoam agents (e.g., polyether polyols such as Foamblast 882 (Emerald Foam Control), Erol DF 204K (Ouvrie PMC), DG436 (ODG Industries, Inc.). KFO 880 (KABO Chemicals, Inc.)), sugar alcohols (e.g., sorbitol, maltitol, lactitol, xylitol), surfactants (e.g., alcohol cthoxylates such as Ncodol 23-6.5 (Shell Chemical LP, Houston, TX) and Lutensol TO65 (BASF)), and anti-redeposition agents (e.g., polyethylene glycol polyesters such as Repel-o-Tex SRP6 (Rhodia, Inc.), Texcare SRN-100 or SRN-170 (Clariant GmbH, Sorez-100 (ISP Corp.)). In some embodiments, the enzyme layer contains a water soluble polymer, such as polyvinyl alcohol or polyethylene glycol.

### Microbial Layer

In some embodiments, the granule of the present disclosure contains a microbial layer comprising one or more fermentation solids containing live microbial cultures. The microbial layer may also contain one or more of a polymer, a sugar, a starch, a salt, and a surfactant.

The microorganism is one or more belonging to any of the genera selected from the group consisting of Bacillus, Paenibacillus, Lactobacillus, Brevibacillus, Escherichia, Gluconobacter, Gluconacetobacter, Acetobacter, Streptococcus, Methylobacterium, Pantoea, Pseudomonas, Sphingomonas, Curtobacterium, Knoellia, Massilla, Pedobacter, Skermanella, Clostridia, Klebsiella, Spirillum, Streptomyces, Coniothyrium, Clonostachys, Achromobacter, Saccharomyces, Hanseniaspora, Trichoderma, Aspergillus, Aureobasidium, Ulocladium, Muscodor, Metarhizium, Beauveria, Paecilomyces, Isaria, or Lecanicillium.

### Core

The granules provided herein generally also comprise a core, consisting of one or more inorganic salts. In some embodiments, the core consists of sodium sulfate, sodium citrate, sodium chloride, calcium sulfate, or a combination thereof. In one embodiment, the core consists of sodium sulfate.

The core of the granules provided herein generally has a diameter of about 100 um to about 250 um, about 150 um to about 250 um, or about 250 um to about 300 um.

### Matrix Cores

The granules provided herein may also comprise a matrix core, in which a matrix core is produced containing dried fermentation solids and optionally binders or fillers. The matrix core may be produced by any drying process known in the art, such as spray-drying, spray-granulation, spray-agglomeration, high-shear granulation, extrusion, pan coating, spheronization, drum-granulation, crystallization, precipation, or prill processes. Such processes are known in the art and are described in U.S. Pat. Nos. 4,689,297 and 5,324,649 (fluid bed processing); EP656058B1 and U.S. Pat. No. 454,332 (extrusion process); U.S. Pat. No. 6,248,706 (granulation, high-shear); and U.S. Pat. Not 6,534,466 (combination process utilizing a fluid-bed core and mixer coating). The bioactive may comprise either an enzyme, to produce an enzyme matrix core, or a live culture, to produce a microbial matrix core. The matrix cores can then be coated with additional coating layers via spray-coating, as previously described.

### Detergent Compositions

The granules provided herein find use in the preparation of compositions containing the bioactive granules, which may be subsequently formed into powders, tablets or other unit dose forms of detergent. Such compositions may contain components suitable for use of the granules in particular applications, such as for use in cleaning (e.g. detergents), textiles, or animal feed.

In some embodiments, bioactive-containing granules as described herein are incorporated into a cleaning composition, such as a detergent, e.g., for laundry or dishwashing use, to provide cleaning performance and/or cleaning benefits. Enzymes suitable for inclusion in a cleaning composition include, but are not limited to, hemicellulases, peroxidases, proteases, cellulases, xylanases, lipases, phospholipases, esterases, cutinases, pectinases, keratinases, reductases, oxidases, phenoloxidases, lipoxygenases, ligninases, pullulanases, tannases, pentosanases, malanases, β-glucanases, arabinosidases, hyaluronidase, chondroitinase, laccases, perhydrolases, and alpha-amylases, or mixtures thereof. A typical combination is a cocktail of conventional applicable enzymes like protease, lipase, cutinase and/or cellulase in conjunction with alpha-amylase.

Adjunct materials may also be included in the cleaning composition, for example, to assist or enhance cleaning performance, for treatment of the substrate to be cleaned, or to modify the aesthetics of the cleaning composition as is the case with perfumes, colorants, dyes or the like. It is understood that such adjuncts are in addition to the bioactive-containing granules as described herein. The precise nature of these additional components, and levels of incorporation thereof, will depend on the physical form of the composition and the nature of the cleaning operation for which it is to be used. Suitable adjunct materials include, but are not limited to, surfactants, builders, chelating agents, dye transfer inhibiting agents, deposition aids, dispersants, enzyme stabilizers, catalytic materials, bleach activators, bleach boosters, preformed peracids, polymeric dispersing agents, clay soil removal/anti-redeposition agents, brighteners, suds suppressors, dyes, perfumes, structure elasticizing agents, fabric softeners, carriers, hydrotropes, processing aids and/or pigments.

A cleaning composition as described herein may comprise a surfactant or surfactant system wherein the surfactant can be selected from nonionic surfactants, anionic surfactants, cationic surfactants, ampholytic surfactants, zwitterionic surfactants, semi-polar nonionic surfactants, and mixtures thereof. A surfactant is typically present at a level of about 0.1% to about 60%, about 1% to about 50% or about 5% to about 40% by weight of the subject cleaning composition.

A cleaning composition as described herein may further comprise one or more detergent builder or builder system. When a builder is used, the subject cleaning composition will typically comprise at least about 1%, about 3% to about 60%, or about 5% to about 40% builder by weight of the subject cleaning composition.

Builders that may be used in the cleaning compositions provided herein include, but are not limited to, the alkali metal, ammonium and alkanolammonium salts of polyphosphates, alkali metal silicates, alkaline earth and alkali metal carbonates, aluminosilicate builders, polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof.

A cleaning composition as described herein may also contain one or more chelating agents. Suitable chelating agents include, but are not limited to, copper, iron and/or manganese chelating agents and mixtures thereof. When a chelating agent is used, the cleaning composition may comprise about 0.1% to about 15%, or about 3.0% to about 10% chelating agent by weight of the subject cleaning composition. Suitable cleaning agents include, but are not limited to, sodium salts of glutamic acid diacetic acid (GLDA), and methylglycinediacetic acid (MGDA).

A cleaning composition as described herein may contain one or more deposition aid. Suitable deposition aids include, but are not limited to, polyethylene glycol, polypropylene glycol, polycarboxylate, soil release polymers such as polytelephthalic acid, and clays such as Kaolinite, bentonite, montmorillonite, atapulgite, illite, bentonite, halloysite, and mixtures thereof.

A cleaning composition as described herein may include one or more dye transfer inhibiting agent. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones, and polyvinylimidazoles, and mixtures thereof. When present in a subject cleaning composition, dye transfer inhibiting agent may be present at levels of about 0.0001% to about 10%, about 0.01% to about 5%, or about 0.1% to about 3% by weight of the cleaning composition.

A cleaning composition as described herein may also contain one or more dispersants. Suitable water-soluble organic dispersants include, but are not limited to, the homo- or co-polymeric acids or their salts, in which the polycarboxylic acid comprises at least two carboxyl radicals separated from each other by not more than two carbon atoms.

Enzymes for use in cleaning compositions can be stabilized by various techniques. Enzymes employed herein can be stabilized, for example, by the presence of water-soluble sources of calcium and/or magnesium ions in the finished compositions that provide such ions to the enzymes.

A cleaning composition as described herein may further include one or more catalytic metal complex. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra (methylenephosphonic acid) and water-soluble salts thereof. Such catalysts are disclosed in U.S. Pat. No. 4,430,243. Manganese-containing catalysts useful herein are known, and are described, for example, in U.S. Pat. No. 5,576,282. Cobalt bleach catalysts useful herein are known, and are described, for example, in U.S. Pat. Nos. 5,597,936 and 5,595,967. Such cobalt catalysts are readily prepared by known procedures, such as taught for example in U.S. Pat. Nos. 5,597,936 and U.S. Pat. No. 5,595,967

The compositions provided herein may also include a transition metal complex of a macropolycyclic rigid ligand—abbreviated as “MRL”. As a practical matter, and not by way of limitation, the compositions and cleaning processes herein can be adjusted to provide on the order of at least one part per hundred million of the active MRL species in the aqueous washing medium, and will often provide about 0.005 ppm to about 25 ppm, about 0.05 ppm to about 10 ppm, or about 0.1 ppm to about 5 ppm, of the MRL in the wash liquor. Suitable transition-metals in a transition-metal bleach catalyst include manganese, iron and chromium. In one embodiment, an MRL is an ultra-rigid ligand that is cross-bridged, such as 5,12-diethyl-1,5,8, 12-tetraazabicyclo[6.6.2]hexadecane. Suitable transition metal MRLs are readily prepared by known procedures, such as taught for example in PCT Application No. WO 00/332601 and U.S. Pat. No. 6,225,464.

The cleaning compositions disclosed herein of can be used to clean a site, including a stain, on a surface or fabric. In some embodiments, at least a portion of the site is contacted with a cleaning composition as described herein, in neat form or diluted in a wash liquor, and then the situs is optionally washed and/or rinsed. Washing includes, but is not limited to, scrubbing, and mechanical agitation. A fabric may comprise most any fabric capable of being laundered in normal consumer use conditions. The disclosed cleaning compositions are typically employed at concentrations of from about 500 ppm to about 15,000 ppm in solution. When the wash solvent is water, the water temperature typically ranges from about 5° C. to about 90° C. and, when the situs comprises a fabric, the water to fabric mass ratio is typically from about 1:1 to about 30:1.

Examples of automatic dishwashing compositions that the enzyme granules provided herein can be used in, include those described in US20130130358, WO2017186579. U.S. Pat. No. 8,962,543, EP2885391, US20170022452, WO2018118745, and US20140018278.

### Methods

Also provided herein are cleaning methods employing the bioactive granules and compositions provided herein.

In one embodiment, methods for cleaning a surface are provided, wherein the method comprises contacting a surface with a cleaning composition comprising a bioactive granule having at least one odor-controlling layer comprising between about 3%-10% of a water-soluble polymer, between about 0.1%-5% of a nonionic surfactant, and between about 0.1%-10% of an odor-controlling compound, such as a zeolite or cyclodextrin.

In some embodiments, the surface to be cleaned in such methods are any surface in need of cleaning. In some embodiments, the surface to be cleaned is a fabric, dish or hard surface.

In another embodiment, methods are provided for reducing the odor associated with a granule containing a fermented product, such as an enzyme or microorganism. Such methods comprise coating a bioactive-contain granule with an odor-controlling layer comprising between about 3%-10% of a water-soluble polymer, between about 0.1%-5% of a nonionic surfactant, and between about 0.1%-10% of an odor-controlling compound, such as a zeolite or cyclodextrin. In some embodiments, the methods provided reduce the odor at least about 10%, 20%, 30%, 35%, 40%, 45%, 50%, 55%, 60%, 65%, 70%, 75%, 80%, 85%, 90%, 95% or more compared to lacking such an odor-controlling layer.

The following examples are provided to demonstrate and illustrate certain preferred embodiments and aspects of the present disclosure and should not be construed as limiting.

### EXAMPLES

### Example 1: Preparation of Enzyme Granules for Odor Analysis

Formulations for 8 batches of granules, all of which were produced via a fluid-bed spray-coating process, are shown below in Tables 1 and 2. To produce the granules, a VFC-LAB1 Flo-Coater (Freund-Vector, Marion, IA, USA) was charged with granules of sodium sulfate denoted as “Cores”. The bed of sodium sulfate cores was fluidized and overcoated with three discrete layers via fluidized-bed coating. The first spray-coated layer, denoted “Layer 1”, comprises enzyme-containing fermentation solids and polyvinyl alcohol (PVA) (Sekisui Specialty Chemical America, Dallas, TX, US). In Granules A-C, the enzyme used was a protease variant as described in WO2012151534 (Genencor International, Inc.). In Granules D-G, the enzyme used was an amylase variant as described in US20080293607 (Genencor International, Inc.). The second spray-coated layer, denoted “Layer 2”, comprises sodium sulfate. The third spray-coated layer, denoted “Layer 3”, comprises titanium dioxide (Tronox, Hamilton, MS, USA), PVA, lutensol (BASF, Ludwigshafen, Germany), and optionally one of the odor absorbers zeolite beta hydrogen (Alfa Aesar, Ward Hill, MA, USA), zeolite beta ammonium (Alfa Aesar), or cyclodextrin (ACROS Organics, Fair Lawn, NJ, USA). Certain details of the fluid-bed process were substantially as described in Example 2 of WO 99/32613, incorporated herein by reference.

TABLE 1
Compositions of protease-containing granules for odor analysis

| | | Granule A | Granule B | Granule C |
|---|---|---|---|---|
| Cores | Sodium Sulfate | 50.2% | 50.6% | 50.7% |
| Layer 1 | Fermentation Solids | 26.8% | 26.3% | 26.4% |
| | PVA | 3.0% | 2.9% | 2.9% |
| Layer 2 | Sodium Sulfate | 8.2% | 8.4% | 8.2% |
| Layer 3 | Titanium dioxide | 5.2% | 2.6% | 2.6% |
| | PVA | 5.2% | 5.2% | 5.2% |
| | Lutensol | 1.4% | 1.4% | 1.4% |
| | Zeolite beta ammonium | | 2.6% | |
| | Zeolite beta hydrogen | | | 2.6% |

TABLE 2
Compositions of amylase-containing granules for odor analysis

| | | Granule | Granule | Granule | Granule |
|---|---|---|---|---|---|
| | | D | E | F | G |
| Cores | Sodium Sulfate | 40.8% | 48.4% | 47.7% | 48.1% |
| Layer 1 | Fermentation Solids | 21.3% | 23.1% | 23.4% | 20.3% |
| | PVA | 2.0% | 2.1% | 2.1% | 1.8% |
| Layer 2 | Sodium Sulfate | 24.1% | 8.7% | 9.1% | 12.1% |
| Layer 3 | Titanium dioxide | 5.2% | 3.9% | 3.9% | 3.9% |
| | PVA | 5.2% | 7.8% | 7.8% | 7.8% |
| | Lutensol | 1.4% | 2.1% | 2.1% | 2.1% |
| | Zeolite beta ammonium | | 3.9% | | 3.9% |
| | Beta-cyclodextrin | | | 3.9% | |

The fermentation solids used to produce Granules A, B, and C were produced from fermentations of B. subtilis that were lysed and concentrated. The fermentation, lysis, and concentration steps were carried out according to identical procedures. Gas chromatography with mass spectrometry (GC-MS) analysis was used to confirm that the concentrates used to produce these granules contained similar quantities of odorous compounds such as short-chain fatty acids, sulfides, and amines. Granule A contains no odor absorber in Layer 3. Granule B contains the odor absorber zeolite beta ammonium in Layer 3. Granule C contains the odor absorber zeolite beta hydrogen in Layer 3.

The fermentation solids used to produce Granules D, E, F, and G were produced from fermentations of B. licheniformis that were lysed and concentrated. The fermentation, lysis, and concentration steps for Granules D, E, and F were carried out according to identical procedures. GC-MS analysis was used to confirm that the concentrates used to produce these granules contained similar quantities of odorous compounds such as short-chain fatty acids, sulfides, and amines. For Granule G, the fermentation and lysis steps were identical to those used for Granules D, E and F; however, the concentration step involved diafiltration of the lysed broth for an additional 1.7 diavolumes. This additional diafiltration had the effect of further concentrating macromolecules, such as enzymes, within the fermentation solids, and removing smaller, potentially odorous molecules. GC-MS analysis was used to confirm reduction in odorous compounds such as short-chain fatty acids, sulfides, and amines. Granule D contained no odor absorber in Layer 3. Granules E and G contained the odor absorber zeolite beta ammonium in Layer 3. Granule F contained the odor absorber beta-cyclodextrin in Layer 3.

### Example 2: Odor Panel Training and Set-Up

Odor panels were conducted to assess the level of malodor in the Granules A, B, C, D, E, F, and G described in Example 1. Granules were packaged in 20-mL scintillation vials (Wheaton, Millville, NJ, USA). A 1-g aliquot of granules was added to each vial. The vial was covered with a 3-ply layer of Kimwipe (Kimberly-Clark, Dallas, TX, USA) to prevent escape of sensitizing enzyme dust, and the vial cap was screwed on. After packaging in the scintillation vials, the granules were aged in an incubator at 48 C for 3 weeks; preliminary testing had shown these aging conditions were effective in simulating storage conditions experienced by the enzyme granules during transit, and also resulted in the generation of the most offensive malodors from the granules.

An odor panel comprising 8 volunteer panelists was assembled. The panel was trained, by providing them with various example products, to identify several malodorous compounds that can arise in fermented products, including short-chain fatty acids, acetic acid, pyrazines, sulfides, and amines. The odor panel was trained to identify differences in intensity of smell by being given aliquots of acetic acid in various concentrations.

The odor panel was presented with the granules in scintillation vials that had been aged for 3 weeks. All vials were identified with a randomly-generated three-digit code rather than with a granule name to minimize bias. Odor panelists were asked to smell the vials in pairs, with one vial assigned as a Control sample and one vial assigned as a Test sample. Each panelist smelled the pairs of vials in a randomized order.

For each pair of vials, odor panelists were asked to evaluate the overall odor intensity of the Test sample compared to the control. The panelists were asked to record the level of difference between the Control sample and the Test sample according to a scale of no difference, slightly different (a difference can be discerned after some time), moderately different (a difference in odor is immediately apparent, with some similarity between the samples), or extremely different (it is immediately clear from the odor that there is an obvious sample difference). For each pair of vials, odor panelists were further asked which sample was preferable from an odor perspective. For each pair of vials, each panelist's level-of-difference and preferred-odor ratings were converted to a numerical value between +3 and −3, as described in Table 3. Panelists were additionally asked to utilize a similar procedure to compare samples according to their perceptions of specific odors on which they were trained, and to provide other notes on odors found in the samples.

TABLE 3
Conversion of odor panelist responses to odor panel

| | Granule with | Numerical |
|---|---|---|
| Level of difference | preferred odor | value |
| Extremely different | Control | +3 |
| Moderately different | Control | +2 |
| Slightly different | Control | +1 |
| No difference | Neither | 0 |
| Slightly different | Test | −1 |
| Moderately different | Test | −2 |
| Extremely different | Test | −3 |

According to the scale shown in Table 3, positive values correspond to results in which an odor panelist preferred the odor of the Control granule, whereas negative values correspond to results in which an odor panelist preferred the odor of the Test granule. A greater absolute value corresponds to a greater level of difference perceived by the odor panelist. For each pair of granules, the numerical results from all of the panelists were averaged and the standard deviation was recorded.

### Example 3: Odor Panels for Protease Granules

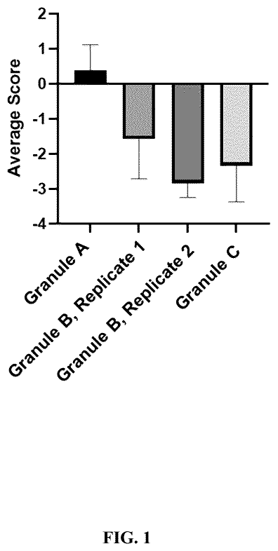

Odor panels were conducted to assess the level of malodor in Granules A, B, and C, according to the method described in Example 2. All vials labeled as Control contained Granule A. Vials labeled as Test contained Granules A, B, and C. Thus, Test vials containing Granules A, B, and C were each compared to Control vials containing Granule A. Two replicates of Granule B were included as Test samples in the odor panels. The numerical results of the odor panelists' surveys were prepared as described in Example 2. The results are shown in FIG. 1.

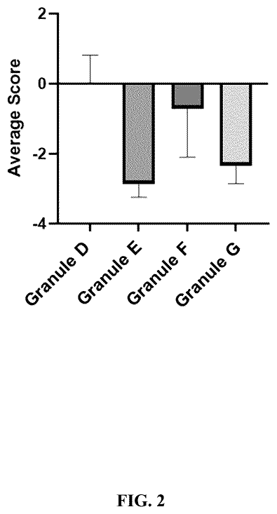

The data in FIG. 2 show that when Granule A was presented as a Test granule, odor panelists described it as having no difference compared to the Control vial, also containing Granule A. This verifies that the odor panelists were able to identify granules with similar levels of odor. When Granule B was presented as a Test granule in two replicates, odor panelists described its odor as slightly to extremely preferable in comparison to Granule A. Granule B included zeolite beta ammonium in its Layer 3, while Granule A included no odor absorbing compounds in any layer. The inclusion of zeolite beta ammonium in Layer 3 of Granule B therefore reduced the odor of the granule. Similarly, odor panelists described the odor of Granule C as moderately to extremely preferable in comparison to Granule A. Granule C included zeolite beta hydrogen in its Layer 3, while Granule A included no odor absorbing compounds in any layer. The inclusion of zeolite beta hydrogen in Layer 3 of Granule C therefore reduced the odor of the granule.

Additional aliquots of granules A, B, and C were analyzed via GC-MS. These aliquots had been aged identically to the granule samples given to the odor panel. Several peaks of known odorous compounds were selected, and the resulting peak areas were reported in the units of counts*min. Table 4 shows the results of this GC-MS analysis.

TABLE 4
GC-MS analysis results for Granules A, B, and C

| | Peak Area (Counts*min) | | |
|---|---|---|---|
| Granule | Isobutyric acid | Isovaleric acid | Dimethyl disulfide |
| Granule A | 141671 | 193561 | 26703 |
| Granule B | 1779 | 2263 | 0 |
| Granule C | 1795 | 2329 | 3486 |

The three compounds identified in Table 4 are malodorous. Isobutyric acid is known to have a rotten dairy smell. Isovaleric acid is known to have a body-odor or cheesy smell. Dimethyl disulfide is known to have a garlicky or rotten-fish smell. In Granules B and C, the peak areas for isobutyric and isovaleric acids detected by GC-MS were reduced by about two orders of magnitude compared to the control Granule A. Also relative to the control Granule A, the peak areas for dimethyl disulfide detected by GC-MS was reduced to zero in the case of Granule B, or by about one order of magnitude for Granule C. This significant reduction in known malodorous compounds corresponds with the findings of the odor panel that the zeolite-containing Granules B and C were less malodorous than the control Granule A.

### Example 4: Odor Panels for Amylase Granules

Odor panels were conducted to assess the level of malodor in Granules D, E, F, and G, according to the method described in Example 2. All vials labeled as Control contained Granule D, which included no odor-controlling compounds. Vials labeled as Test contained Granules D, E, F, and G. Thus, Test vials containing Granules D, E, F, and G were each compared to Control vials containing Granule D. The numerical results of the odor panelists' surveys were prepared as described in Example 2. The results are shown in FIG. 2.

The data in FIG. 2 show that when Granule D was presented as a Test granule, odor panelists described it as having no difference compared to the Control vial, also containing Granule D. This verifies that the odor panelists were able to discern amylase granules with similar odors. When Granule E was presented as a Test granule, odor panelists described its odor extremely preferable in comparison to Granule D. Granule E included zeolite beta ammonium in its Layer 3, while Granule D included no odor absorbing compounds in any layer. The inclusion of zeolite beta ammonium in Layer 3 of Granule E therefore reduced the odor of the granule. Similarly, when Granule F was presented as a Test granule, odor panelists described its odor slightly preferable in comparison to Granule D. Granule F included cyclodextrin in its Layer 3, while Granule F included no odor absorbing compounds in any layer. The inclusion of cyclodextrin in Layer 3 of Granule F therefore reduced the odor of the granule. Odor panelists also described the odor of Granule G as moderately preferable in comparison to Granule D. Granule G included zeolite beta ammonium in its Layer 3, while Granule D included no odor absorbing compounds in any layer. Granule G also incorporated diafiltration into its production process in order to reduce the odor of the granule. However, overall, the level of odor reduction in Granule G was not greater than the level of odor reduction achieved in Granule E, which was produced without this additional diafiltration step. The inclusion of zeolite beta ammonium in Layer 3 of Granules E and G is therefore a more effective method than diafiltration for reducing the malodor of the granule.

Additional aliquots of granules D, E, F, and G were analyzed via GC-MS. These aliquots had been aged identically to the granule samples given to the odor panel. Several peaks of known odorous compounds were selected, and the resulting peak areas were reported in the units of counts*min. Table 5 shows the results of this GC-MS analysis.

TABLE 5
GC-MS analysis results for Granules D, E, F, and G

Peak Area (Counts*min)

| Granule | Isobutyric acid | Isovaleric acid |
|---|---|---|
| Granule D | 55520 | 138510 |
| Granule E | 16892 | 12504 |
| Granule F | 32184 | 78967 |
| Granule G | 19512 | 9374 |

The two compounds identified in Table 4 are malodorous. Isobutyric acid is known to have a rotten dairy smell. Isovaleric acid is known to have a body-odor or cheesy smell. Dimethyl disulfide is known to have a garlicky or rotten-fish smell. In Granules E, F, and G, the peak areas for isobutyric and isovaleric acids detected by GC-MS were reduced by about 40-90% relative to those control Granule D. This significant reduction in known malodorous compounds corresponds with the findings of the odor panel that Granules E, F, and G, all of which contain the odor-controlling compounds zeolite or cyclodextrin, were less malodorous than the control Granule D. Of Granules E, F, and G, the least reduction in GC-MS peak area for these two compounds was observed for Granule F, which contained cyclodextrin. This correlates with the odor panel's observation that the magnitude of odor reduction in Granule F was less than that of Granules E and G.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

1. A granule comprising a core and at least one layer, wherein the granule comprises:
a. at a first layer comprising about 0.1% to about 40% wt/wt of bioactive enzyme or microorganism; and
b. an odor-controlling coating layer surrounding the first layer, wherein the odor-controlling coating layer comprises between about 3-10% wt/wt of a water-soluble polymer, between about 0.1%-5% wt/wt of a nonionic surfactant, and between about 0.1-10% wt/wt of an odor-controlling compound.

2. The granule of claim 1, wherein the enzyme is selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, DNase or nuclease, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, lysozymes, mannanases, oxidases, pectate lyases, pectin acetyl esterases, pectinases, pentosanases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, xylosidases, metalloproteases, nucleases, serine proteases, and combinations thereof.

3. The granule of claim 1, wherein the enzyme comprises a protease, an amylase, or a combination of a protease and an amylase.

4. The granule of claim 1, wherein the water-soluble polymer is a polyvinyl polymer selected from the group consisting of PVP, PVA, and copolymers thereof.

5. The granule of claim 1, wherein the odor-controlling coating layer comprises between about 4-6% wt/wt of a water-soluble polymer.

6. The granule of claim 1, wherein the nonionic surfactant is an alcohol ethoxylate.

7. The granule of claim 1, wherein the odor-controlling layer comprises between about 1%-3% wt/wt of a nonionic surfactant.

8. The granule of claim 1, where the odor-controlling compound comprises a zeolite, a cyclodextrin, a saccharide or functionalized saccharide, a clay, a metal oxide, a metal carbonate, a metal-organic framework, activated carbon, or combinations thereof.

9. The granule of claim 8, wherein the zeolite is selected from the group consisting of zeolite-beta ammonium or zeolite-beta hydrogen.

10. The granule of claim 8, wherein the cyclodextrin is selected from the group consisting of alpha-cyclodextrin, beta-cyclodextrin, and gamma-cyclodextrin.

11. The granule of claim 8, wherein the saccharide or functionalized saccharide is selected from the group consisting of starch, cellulose, methylcellulose, and hydroxypropylmethylcellulose.

12. The granule of claim 8, wherein the clay is selected from the group consisting of kaolinite, bentonite, montmorillonite, atapulgite, illite, bentonite, halloysite or diatomite.

13. The granule of claim 8, wherein the metal oxide is selected from the group consisting of titanium dioxide, magnesium oxide, iron (III) oxide, calcium oxide, or silicon dioxide.

14. The granule of claim 8, wherein the metal carbonate is selected from the group consisting of calcium carbonate, magnesium carbonate, or sodium hydrogen carbonate.

15. The granule of claim 8, wherein the metal-organic framework is selected from the group consisting of MOF-5, MOF-177, or MOF-199.

16. The granule of claim 8, wherein the odor-controlling compound is a zeolite and comprises about 2.5% of the total granule composition.

17. The granule of claim 8, wherein the odor-controlling compound is a cyclodextrin and comprises about 3.9% of the total granule composition.

18. A granule comprising a core and at least one odor-controlling layer, wherein the granule comprises:
a. a bioactive matrix core comprising about 0.1% to about 40% wt/wt of bioactive enzyme or microorganism; and
b. an odor-controlling coating layer surrounding the bioactive matrix core, wherein the odor-controlling coating layer comprises between about 3-10% wt/wt of a water soluble polymer, between about 0.1%-5% wt/wt of a nonionic surfactant, and between about 0.1-10% wt/wt of an odor-controlling compound.

19-34. (canceled)

35. A method of cleaning a surface, the method comprising:
a. contacting a surface with a composition comprising a granule of claim 1 or claim 18; and,
b. rinsing the surface.

36. (canceled)

37. A method of reducing the odor associated with a granule comprising a bioactive enzyme or microbial obtained from a fermentation process by coating with an odor-controlling layer, wherein the odor-controlling layer comprises a water soluble polymer, a nonionic surfactant, and an odor-controlling compound, such as a zeolite, a cyclodextrin, a saccharide or functionalized saccharide, a clay, a metal oxide, a metal carbonate, a metal-organic framework, or activated carbon.

38-48. (canceled)
